(19) United States
(12) Patent Application Publication
(10) Pub. No.: US 2026/0335132 A1
(43) Pub. Date: Oct. 29, 2026

(54) SURGICAL APPARATUS AND METHOD OF USE

(71) Applicant: NALLAKRISHNAN FAMILY TRUST, Willowbrook, IL (US)

(72) Inventor: Ravi Nallakrishnan, Willowbrook, IL (US)

(21) Appl. No.: 19/113,613

(22) PCT Filed: Sep. 5, 2023
(86) PCT No.: PCT/US2023/031958

§ 371 Date: Mar. 20, 2025

Related U.S. Application Data
(60) Provisional application No. 63/417,781, filed on Oct. 20, 2022.

(51) Int. Cl. A61F 9/007

(52) U.S. Cl. CPC A61F 9/00736; A61B 2217/005; A61B 2217/007

(57) ABSTRACT

An ophthalmic surgical apparatus (40) for use with an instrument includes a hollow terminal portion (50) defining an internal passage (54) and at least one aspiration port (56) and a hollow sheath portion (70) extending from the terminal portion (50) along a central axis and defining at least one irrigation port (74). The sheath portion (70) defines a first external diameter in a direction perpendicular to the central axis. The apparatus (40) includes a flaring, hollow hub portion (90) extending from the sheath portion (70). The hub portion (90) defines an open proximal end (94) for receiving the instrument. The hub portion (90) defines an internal connecting means (98) to secure the hub portion (90) around the instrument. The hub portion (90) defines a second external diameter that is greater than the first external diameter of the sheath portion (70).

### PRIORITY

This application claims priority to U.S. provisional application Ser. No. 63/417,781, filed Oct. 20, 2022, the entire contents of which is incorporated herein by reference.

### FIELD OF THE INVENTION

The present invention relates generally to surgical apparatuses used in ophthalmic surgery and, more particularly, to an improved irrigation and aspiration surgical apparatus and method of use thereof.

### BACKGROUND OF THE INVENTION

Phacoemulsification has come to be a technique of choice for the removal of damaged or diseased lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. The lens of the eye is held within a capsular bag positioned behind the iris in the anterior chamber of the eye. When the lens becomes damaged or diseased, a common surgical technique is to remove the lens and replace it with an artificial intraocular lens (IOL). Removal of the lens is commonly carried out by physical dismemberment of the lens and phacoemulsification, that is, using an instrument to which vibrational energy is transmitted by a phacoemulsification hand piece. The natural lens may also be removed by way of femtosecond laser which softens or dismembers the lens.

During phacoemulsification, the lens is broken into fragments and the fragments are emulsified and then removed from the capsular bag by aspirating the fragments through a cannula formed as part of the phacoemulsification instrument. After the lens has been removed, the capsular bag must be cleaned in order to prepare it for the insertion of an IOL. In particular, epithelial, and cortical tissue fragments must be removed from the floor and perimeter of the capsular bag. Typically, an irrigation and aspiration tip is coupled to a hand piece, which may be the phacoemulsification hand piece, for removal of the fragments. The hand piece will typically have an irrigation and aspiration mode specifically for this task. U.S. Patent Application Publication No. US 2016/037854 A1, hereby incorporated by reference in its entirety, discloses an irrigation and aspiration apparatus have any enlarged, ball-like tip with one or more irrigation or aspiration ports, and a sheath portion that locks into a recess on an outer surface of a metal cannula. Complete removal of the cortex is important for several reasons. If the cortex is not completely removed, it may cause post-operative inflammation and an increase in intraocular pressure. Incomplete removal of the cortex may also cause decentration or tilting of the IOL which, in turn, would cause a postoperative refractive error or induced astigmatism. This is especially important if the IOL is a multi-focal or toric type. Incomplete removal of the cortex may also result in the formation of after (secondary) cataract which would impair vision and require laser capsulotomy.

The inventor has found that the changing of phacoemulsification tips with specialized irrigation and aspiration tips in a handpiece can be inefficient during a surgical procedure. Accordingly, there remains a need for a user friendly apparatus that may be used with commercially available, conventional phacoemulsification tips to facilitate an irrigation and aspiration procedure, and which are specifically configured to promote safe and efficient irrigation and aspiration of the capsular bag.

### SUMMARY OF THE INVENTION

The present invention is directed to an improved surgical apparatus which is particularly suited for use with an associated cannula operative connected to an irrigation and aspiration handpiece.

In accordance with one broad form of the present invention, the apparatus has the form of a hollow terminal portion defining an internal passage communicating with at least one aspiration port in the terminal portion to permit fluid communication through the terminal portion. The apparatus further includes a hollow sheath portion extending along a central axis from the terminal portion. The sheath portion defines at least one irrigation port and a first external diameter in a direction perpendicular to the central axis. The apparatus further includes a flaring, hollow hub portion extending from the sheath portion, and which defines an open proximal end for receiving an instrument operatively connected to an irrigation fluid source and/or a vacuum source. The hub portion has an internal connecting means to secure the hub portion around the instrument received within the open proximal end. The hub portion defines a second external diameter in a direction perpendicular to the central axis that is greater than the first external diameter of the sheath portion.

In one form of the present invention, the apparatus is in combination with an instrument in the form of a cannula operably connected to a handpiece this is operatively connected to a vacuum source and an irrigation fluid source.

In one preferred form of the present invention, the cannula is formed of metal and seals against an internal surface in the terminal portion to establish an aspiration passageway through the terminal portion and an interior of the cannula. Furthermore, the sheath portion is formed from a polymer and is spaced from an exterior surface of the cannula to establish an irrigation fluid passageway surrounding the cannula.

In yet another preferred form of the present invention, the terminal portion has a substantially spherical shape defining a third external diameter in a direction perpendicular to the central axis that is greater than the first external diameter of the sheath portion.

In yet another preferred form of the present invention, the terminal portion has a substantially spherical shape defining a third external diameter in a direction perpendicular to the central axis that is less than the second external diameter of the hub portion.

In still another preferred form of the present invention, the terminal portion has a substantially spherical shape defining a third external diameter in a direction perpendicular to the central axis that is less than the second external diameter of said hub portion.

In yet another preferred form of the present invention, the hub portion has a frustoconical upper end connecting to the sheath portion.

According to one preferred form of the present invention, the connecting means of the hub portion is an internal thread within the hub portion.

According to one preferred form of the present invention, the terminal portion, the sheath portion, and the hub portion are unitarily formed from a polymer.

In another form of the present invention, the terminal portion of the tip has a spherical configuration. Preferably, the polymer is an elastomer.

In another form of the present invention, the sheath portion includes a pair of oppositely-facing irrigation ports. Preferably, the ports are tapered in a direction along the central axis moving toward the terminal portion and have a non-tapered semi-circular shape in a direction along the central axis moving toward the hub portion.

In another preferred form of the present invention, the aspiration port is angled between about 40 degrees and about 50 degrees relative to the central axis.

According to one preferred form of the present invention, the terminal portion defines an outer surface that is relatively rougher than the sheath portion.

In one aspect of the present invention, the terminal portion defines at least one recess, protrusion, and/or indicia therein for indicating the location of the at least one aspiration port.

According to another aspect of the present invention, the terminal portion further includes an annular internal abutment extending into the internal passage and spaced from a terminal end surface of the terminal portion for retaining a cannula at a predetermined distance from the terminal end surface of the terminal portion.

In accordance with another broad form of the present invention, a method of using the surgical apparatus of the present invention is contemplated. The method includes the step of obtaining the apparatus as set forth in any of the embodiments discussed above. The method includes the step of assembling the apparatus with a cannula (such as a phacoemulsification needle) that is operably connected to a handpiece (such as a vibratory handpiece) that itself is operatively connected to a vacuum source and/or an irrigation fluid source to establish an aspiration passageway through the terminal portion of the surgical apparatus and an interior of the cannula and an irrigation fluid passageway surrounding an exterior surface of the cannula and the sheath portion of the surgical apparatus. The method includes the step of aspirating material at a surgical site through the aspiration passageway. The method includes the step of introducing an irrigation fluid to a surgical site through the irrigation fluid passageway. The method may include the further step of removing the surgical apparatus from around the cannula and replacing it with a standard infusion sleeve whereby a distal end of the cannula protrudes from beyond the end of the infusion sleeve.

### BRIEF DESCRIPTION OF THE DRAWINGS

In the accompanying drawings forming part of the specification, in which like numerals are employed to designate like parts throughout the same,

FIG. 1 is an enlarged, isometric view from above of a first illustrated embodiment of an irrigation and aspiration apparatus of the present invention for being assembled at the distal end of a cannula for being attached to a handpiece that is connected to a vacuum source and an irrigation fluid supply source;

FIG. 2 is an enlarged, side elevation view of the apparatus of FIG. 1;

FIG. 3 is an enlarged, front elevation view of the apparatus of FIG. 1;

FIG. 4 is an enlarged, bottom plan view of the apparatus of FIG. 1;

FIG. 5 is a greatly enlarged, fragmentary, detailed view of the terminal portion of the apparatus of FIG. 5;

FIG. 6 is a greatly enlarged, fragmentary, detailed view of an irrigation port of the sheath portion of the apparatus of FIG. 6;

FIG. 7 is an enlarged, cross-sectional view of the apparatus of FIG. 1, taken along the along a vertical plane 7-7 in FIG. 3 extending through a central axis of the apparatus;

FIG. 8 is an enlarged, cross-sectional view of the apparatus of FIG. 1 assembled with a fragmentary portion of an aspiration cannula and an irrigation cannula with the irrigation fluid supply source and vacuum source shown diagrammatically;

FIG. 9 is an enlarged, cross-sectional view of a second illustrated embodiment of an irrigation and aspiration apparatus of the present invention for being assembled at the distal end of a cannula for being attached to a handpiece that is connected to a vacuum source and an irrigation fluid supply source, and FIG. 9 is taken along a vertical plane extending through a central axis of the apparatus;

FIG. 10 is a greatly enlarged cross-sectional view of the tip portion of the apparatus of FIG. 9 showing the internal abutment ring; and

FIG. 11 is an enlarged, cross-sectional isometric view of the irrigation and aspiration apparatus of FIG. 12 assembled with a cannula having a flared tip, and

FIG. 12 is taken along a vertical plane extending through a central axis of the apparatus and the needle.

### DETAILED DESCRIPTION OF THE PREFERRED EMBODIMENTS

While the present invention is susceptible of embodiment in various forms, there are shown in the drawings and will hereinafter be described the presently preferred embodiments, with the understanding that the present disclosure should be considered as an exemplification of the invention, and is not intended to limit the broadest forms of the invention to only the specific embodiments illustrated.

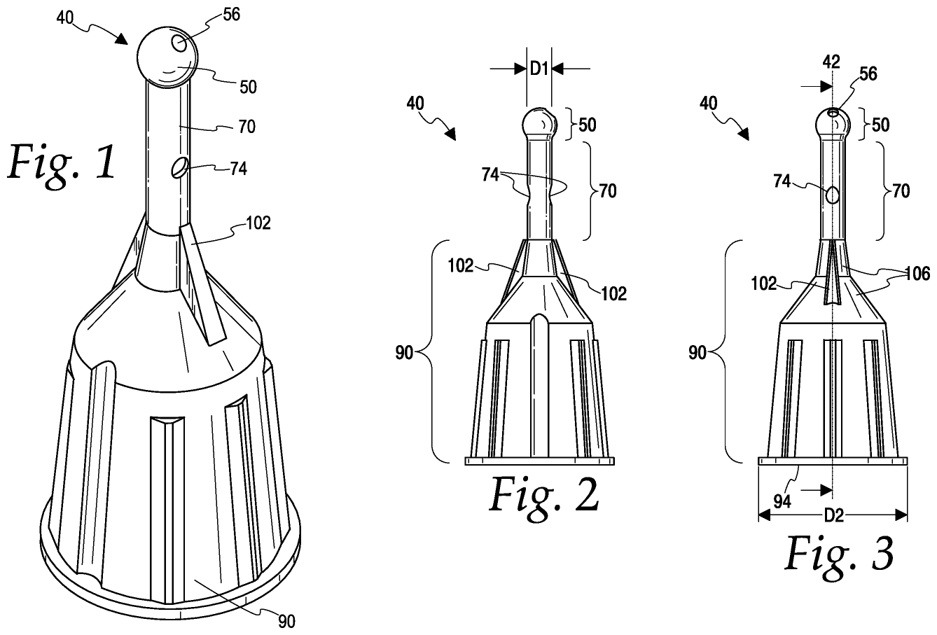

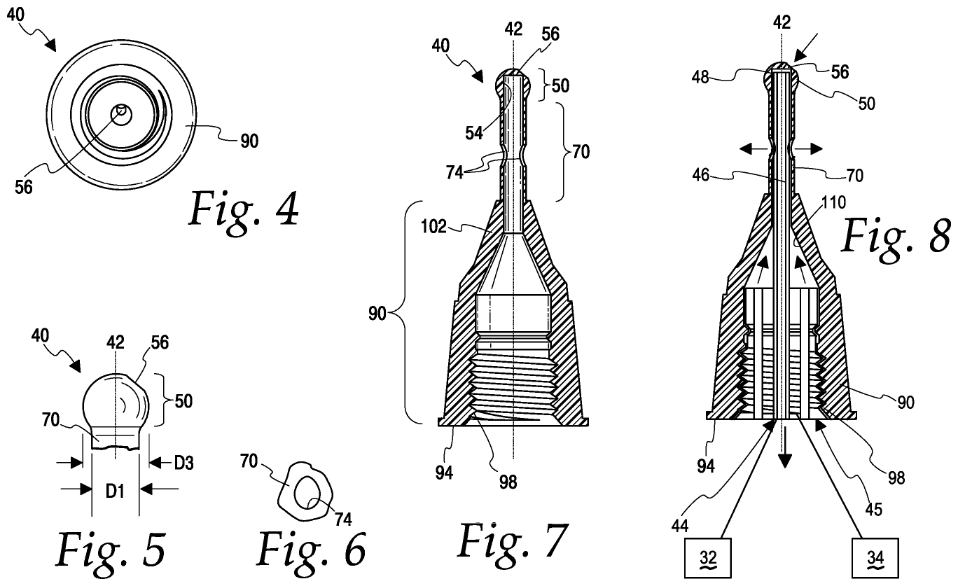

A first illustrated embodiment of an ophthalmic surgical apparatus 40 according to the present invention is shown in FIGS. 1-8, wherein the apparatus 40 is configured for being assembled with a hollow aspiration cannula, needle, or shaft 44 (FIG. 8 only) for being removably connected to an irrigation cannula or handpiece 45 (FIG. 8 only) operably connected to an irrigation fluid supply 34 and a vacuum source 32. The cannula 44, which may be a phacoemulsification tip or needle, defines a lumen or an internal passage 46 through which aspiration is effected during surgery when the cannula 44 is connected to a vacuum source 32 via the handpiece 45. It will be understood that the cannula 44 may have any shape or structure (straight, angled, etc.) and may have a variety of means or structures suited for being attached to a variety of handpieces, such as by mating screw threads, clamps, locks, friction fitting, etc. Suitable structures of instruments are described in U.S. Pat. No. 8,764,782, the entirety of which is incorporated herein by reference. The cannula 44 is preferably formed from a sufficiently rigid metal or hard thermoplastic material. Other materials may be used.

Referring now to FIGS. 7 and 8, the apparatus 40 defines a central axis 42 and is hollow for being attached around a distal end 48 of the cannula 44 (visible in FIG. 8). Importantly, the apparatus 40, and its portions or sections mentioned herein, is unitarily formed from a polymer, preferably an elastomer such as silicone, which is substantially softer and less rigid than the material of the cannula 44 (which may be metal or a rigid or hard polymer) to permit the apparatus 40 to safely contact the lens capsule during use.

With reference to FIG. 7, the apparatus 40 includes a distal, hollow terminal portion 50 that has a rounded or substantially spherical configuration and that defines an internal aspiration passage 54 terminating in an aspiration port 56. While the port 56 and associated internal passage 54 have a circular cross-sectional configuration, taken along the central axis thereof in the direction of flow of aspirated material, it will be understood that the aspiration port 56 may have cross-sectional shapes other than circular, such as polygonal, oval, conical, or tapering up or tapering down, etc. Furthermore, it will be understood that the terminal portion 50 may be provided with more than one aspiration port 56, and such plurality of aspiration ports 56. While the illustrated preferred embodiment of the terminal portion 50 is substantially spherical in shape, other shapes are contemplated, such as tapered, conical, or bullet shaped, polygonal or irregular shaped terminal portions (not illustrated).

As will be discussed in greater detail, the terminal portion internal aspiration passage 54 fluidly connects with the distal end 48 of the cannula 40 to establish an aspiration pathway to facilitate aspiration of fluids and tissues through the apparatus 40 when the cannula proximal end is subjected to a vacuum. The aspiration port 56 is angled about 45 degrees relative to the central axis 42 (as can be seen in FIGS. 5 and 7). It will be understood that the aspiration port 56 may be located at any angle along a 360 degree sector on the terminal portion 50. For example, the port 56 may be aligned with the central axis 42 or angled 20 degrees or 120 degrees relative to the central axis 42.

A substantially cylindrical hollow sheath portion 70 extends from the terminal portion 50 along the central axis 42. The sheath portion 70 includes a pair of opposing irrigation ports 74 through the thickness of the wall of the sheath portion 70. The sheath portion 70 defines a first diameter D1 in the radial direction (i.e., perpendicular) relative to the central axis 42. While the irrigation ports 74 have an ovoid or egg-shaped cross-sectional configuration, taken along the central axis thereof, it will be understood that the irrigation ports 74 may have other cross-sectional shapes such as circular, polygonal, oval, conical, or tapering up or tapering down, etc. Furthermore, it will be understood that the sheath portion 50 may be provided with only one irrigation port 74 or more than two irrigation ports 74 depending on the application. The location of the irrigation ports 74 may be offset in a direction along the central axis 42. In the preferred form of the invention as illustrated, the irrigation ports 74 are located within the same vertical plane as the aspiration port 56 of the terminal portion 50.

With reference to FIGS. 7 and 8, a hollow hub portion 90 flares radially outwardly from the sheath portion 70 in the proximal direction away from the distal, terminal portion 50. The hub portion 90 defines an open proximal end 94 for receiving the cannula 44. The hub portion 90 has an internal connecting means 98 in the form of an internal thread to secure the apparatus 40 around the cannula 44 in a liquid tight manner, as will be discussed in greater detail hereinafter. While the connecting means 98 is preferably an internal screw thread, other removable means may be employed such as clamps, locks, friction fitting, snap fit beads, etc.

With reference to FIG. 3, the hub portion 90 defines a second external diameter D2, in the direction perpendicular to the central axis 42, that is substantially greater (i.e., at least 1.5 times greater) than the first external diameter D1 of the sheath portion 70. The connection between the hub portion 90 with the sheath portion 70 has the form of a frustoconical upper end 102. A pair of oppositely-facing ribs 106 are aligned in the same vertical plane as the irrigation ports 74 and the aspiration port 50 serve to indicate to a user the location of the ports, which may be obscured or not visible during use of the apparatus 40.

With reference to FIG. 5, terminal portion 50 of the apparatus 40 defines a third external diameter D3, in the direction perpendicular to the central axis 42, which is greater than the first external diameter D1 of the sheath portion 70 but less than the second external diameter D2 of the hub portion 90. The third external diameter D3 of the terminal portion 50 is preferably more than 10% greater in length than the length of the first diameter D1 of the sheath portion 70.

When the apparatus 40 is assembled with the cannula 44, the distal end of the cannula 44 abuts and seals against an internal end surface of the terminal portion 50 to establish an aspiration passage 54 through the terminal portion 50 and the cannula 44 to facilitate aspiration of fluids and tissues through the apparatus 40 when the cannula 44 interior is subjected to a vacuum. Furthermore, an irrigation fluid passage 110 is established within the hollow hub portion 90 around the exterior of the cannula 44 and terminating at the irrigation fluid ports 74 to facilitate delivery of irrigation fluids through the apparatus 40 when subjected to an irrigation fluid from a fluid supply source.

The outer surface of the terminal portion 50 may define one or more recesses or protrusions (not illustrated) arranged to further indicate to a user the location of the ports 56 and/or 74. Furthermore, the recesses or protrusions in the terminal portion 50 may function to prevent or at least minimize the adhesion of the emulsified nucleus during use of the apparatus 40.

It is contemplated that the outer surface of the terminal portion 50 may have a smooth or a roughened surface (rougher than that of the sheath portion 70) for polishing of the lens capsule.

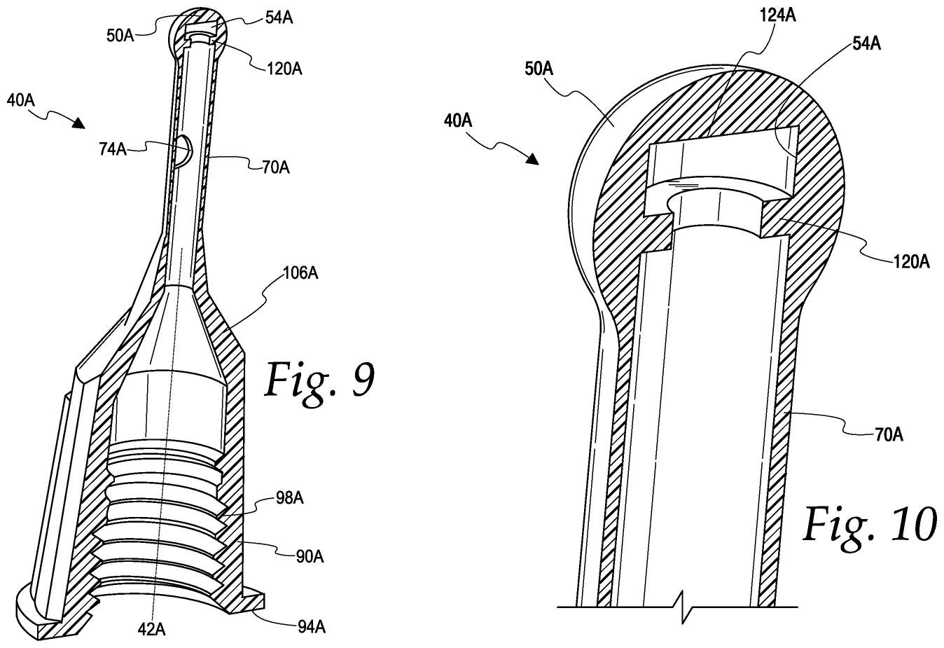

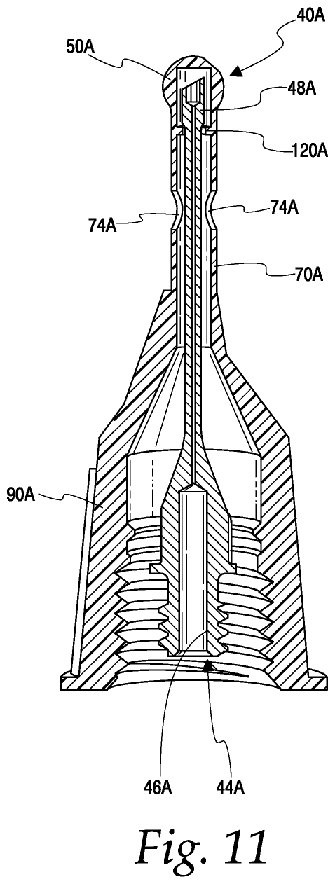

A second embodiment of a surgical apparatus or instrument according to the present invention is shown in FIGS. 9-11, is designated by the numeral 40A, and functions similarly to the first illustrated embodiment of the apparatus 40 as previously described in FIGS. 1-8. The numbered features of the second embodiment of the apparatus 40A illustrated in FIGS. 9-11 are analogous to features of the first embodiment of the apparatus 40 that share the same number, but without the suffix “A”. The second embodiment of the apparatus 40A includes the same basic features of a distal, hollow terminal portion 50A that has a rounded or substantially spherical configuration and that defines an internal aspiration passage 54A terminating in an aspiration port. The apparatus 40A further includes a substantially cylindrical hollow sheath portion 70A extends from the terminal portion 50A along a central axis 42A. The sheath portion 70A includes a pair of opposing irrigation ports 74A through the thickness of the wall of the sheath portion 70A. The apparatus 40A further includes a hollow hub portion 90A flares radially outwardly from the sheath portion 70A in the proximal direction away from the distal, terminal portion 50A. The hub portion 90A defines an open proximal end 94A for receiving a cannula 44. The hub portion 90A has an internal connecting means in the form of an internal thread or threads to secure the apparatus 40A around the cannula 44 or 44A in a liquid tight manner.

As best seen in FIG. 10, the apparatus 40A differs from the prior-discussed embodiment 40 of the present invention in that the inside of the passageway 54A includes one or more projections or abutments 120A to maintain the distal end 48A of the cannula 44A at a predetermined location within the apparatus 40A. The preferred embodiment of the abutment 120A has the form of an annular projection extending fully around the circumference of the inside of walls of the passage 54A to prevent the distal end 48A of the cannula 44A from contacting the internal end surface 124A of the terminal portion 50A of the apparatus 40A when assembled. Accordingly, when the apparatus 40A is assembled with the cannula 44A, the distal end 48A of the cannula 44A is locked in position and is spaced from the internal end surface 124A of the terminal portion 50A to prevent any damage to the elastomeric portions of the terminal portion 50A of the apparatus 40A by the metal cannula 44A for improved safety in the eye. The locking of the cannula 44A within the terminal portion 50A further maximizes the aspiration effect of the apparatus 50A by preventing obstruction between the internal surfaces of the terminal portion 50A and the lumen of the cannula 44A. The particular cannula 44A illustrated in FIG. 12 has a flared distal end or tip 48A that is maintained at a predetermined location within the terminal portion 50A by the annular abutment 120A acting on the portion of the cannula 44A proximal to the flared tip 48A.

The inventor has found that the inventive apparatus is especially beneficial in use of a conventional phacoemulsification needle or tip for aspiration of the cortex while simultaneously keeping polishing the posterior capsule without requiring the removal of the phacoemulsification needle or tip from the handpiece. Furthermore, the apparatus of the present invention may be cost effective in that it eliminates the need for the purchase of a specialized irrigation and aspiration needle or tip.

From the foregoing, it will be observed that numerous modifications and variations can be effected without departing from the true spirit and scope of the novel concept of the present invention. It is to be understood that no limitation of the broadest concepts with respect to the specific embodiments illustrated herein is intended or should be inferred. The disclosure is intended to cover, by the appended claims, all such modifications as fall within the scope of the claims.

1. An ophthalmic surgical apparatus, said apparatus comprising:
a hollow terminal portion defining an internal passage communicating with at least one aspiration port in said terminal portion to permit fluid communication through said terminal portion;
a hollow sheath portion extending along a central axis from said terminal portion, said sheath portion defining at least one irrigation port, said sheath portion defining a first external diameter in a direction perpendicular to said central axis; and
a flaring, hollow hub portion extending from said sheath portion, said hub portion defining an open proximal end for receiving an instrument operatively connected to an irrigation fluid source and/or a vacuum source, said hub portion defining internal connecting means to secure said hub portion around the instrument, said hub portion defining a second external diameter in a direction perpendicular to said central axis that is greater than said first external diameter of said sheath portion.

2. The ophthalmic surgical apparatus in accordance with claim 1 in combination with an instrument in the form of a cannula operably connected to a handpiece operatively connected to a vacuum source and an irrigation fluid source.

3. The ophthalmic surgical apparatus in accordance with claim 2 wherein said cannula is formed of metal and seals against an internal surface in said terminal portion to establish an aspiration passageway through said terminal portion and an interior of said cannula, said sheath portion is formed from a polymer and is spaced from an exterior surface of said cannula to establish an irrigation fluid passageway surrounding said cannula.

4. The ophthalmic surgical apparatus in accordance with claim 1, wherein said terminal portion has a substantially spherical shape defining a third external diameter in a direction perpendicular to said central axis that is greater than said first external diameter of said sheath portion.

5. The ophthalmic surgical apparatus in accordance with claim 1, wherein said terminal portion has a substantially spherical shape defining a third external diameter in a direction perpendicular to said central axis that is less than said second external diameter of said hub portion.

6. The ophthalmic surgical apparatus in accordance with claim 1, wherein said hub portion has a frustoconical upper end connecting to said sheath portion.

7. The ophthalmic surgical apparatus in accordance with claim 1, wherein said connecting means is an internal thread within said hub portion.

8. The ophthalmic surgical apparatus in accordance with claim 1, wherein said terminal portion, said sheath portion, and said hub portion are unitarily formed from a polymer.

9. The ophthalmic surgical apparatus in accordance with claim 8, wherein said polymer is an elastomer.

10. The ophthalmic surgical apparatus in accordance with claim 1, wherein said sheath portion includes a pair of oppositely-facing irrigation ports.

11. The ophthalmic surgical apparatus in accordance with claim 1, wherein said at least one aspiration port is angled between about 40 degrees and about 50 degrees relative to said central axis.

12. The ophthalmic surgical apparatus in accordance with claim 1, wherein said terminal portion defines an outer surface that is relatively rougher than an outer surface of said sheath portion.

13. The ophthalmic surgical apparatus in accordance with claim 1, wherein said terminal portion defines at least one recess, protrusion, and/or indicia therein for indicating the location of said at least one aspiration port.

14. The ophthalmic surgical apparatus in accordance with claim 1, wherein said terminal portion further comprises an annular internal abutment extending into said internal passage and spaced from a terminal end surface of said terminal portion.

15. A method of using a surgical apparatus, said method comprising the steps of:
obtaining the surgical apparatus according to claim 1;
assembling said apparatus with a cannula operably connected to a handpiece operatively connected to a vacuum source and/or an irrigation fluid source to establish an aspiration passageway through said terminal portion and an interior of said cannula and an irrigation fluid passageway surrounding an exterior surface of said cannula and said sheath portion;
aspirating material at a surgical site through said aspiration passageway; and
introducing an irrigation fluid to a surgical site through said irrigation fluid passageway.

16.-30. (canceled)
